(19) United States
(12) Patent Application Publication
(10) Pub. No.: US 2006/0240492 A1
(43) Pub. Date: Oct. 26, 2006

(54) Carbon nanotube based immunosensors and methods of making and using

(76) Inventors: James F. Rusling, Storrs, CT (US); Fotios Papadimitrakopoulos, Vernon, CT (US)

Correspondence Address: CANTOR COLBURN, LLP, 55 GRIFFIN ROAD SOUTH, BLOOMFIELD, CT, 06002, US

(21) Appl. No.: 11/272,467

(22) Filed: Nov. 11, 2005

Related U.S. Application Data
(60) Provisional application No. 60/627,220, filed on Nov. 12, 2004.

(51) Int. Cl. G01N 33/574

(52) U.S. Cl. 435/7.23; 435/7.92; 977/900

(57) ABSTRACT

An immunoassay device comprises a plurality of carbon nanotubes having a first end and a second end, wherein the nanotubes are aligned substantially parallel relative to one another; a substrate responsive to an electrochemical signal, the substrate being attached to the first end of at least a portion of the plurality of nanotubes; and a capture antibody attached to at least a portion of the nanotubes not at the first end. An immunoassay method comprises providing the disclosed immunoassay device, contacting the immunoassay with a test sample under conditions suitable for binding of an analyte to the capture antibody, wherein binding of the analyte generates, directly or indirectly, an electrochemical signal and detecting the signal. Methods of making the disclosed immunoassay device are also disclosed.

### CROSS REFERENCE TO RELATED APPLICATIONS

This application claims priority to U.S. Provisional Application Serial No. 60/627220 filed on Nov. 12, 2004, which is incorporated in its entirety by reference herein.

### STATEMENT REGARDING FEDERALLY SPONSORED RESEARCH

The U.S. Government has certain rights in this invention pursuant to U.S. Army Research Office Grant No. DAAD-02-1-0381.

### BACKGROUND

Detection and quantitation of proteins and their binding partners are critical for the progress of biomedical research. Modem applications include medical diagnostics, elucidation of disease vectors, immunology, new drug development and emerging fields such as proteomics and systems biology. Diagnosis and treatment of pathogen-related human diseases often rely on binding of toxins or bacteria to antibodies. Antigen-antibody binding can be used to detect a wide variety of proteins and pathogens in biological and environmental samples, such as blood serum, water, aerosols, and food. Measurement of collections of protein cancer biomarkers via immunological approaches is promising for reliable early cancer detection. Detection of suites of biomarkers for a given cancer provides much more reliable diagnostics than a single biomarker. However, accurate measurements of multiple proteins with arrays is at an early stage of development. A few commercial immunoassays, for example, provide very good detection limits for proteins in biological samples but can only analyze a single protein type per sample. Nonetheless, there remains a need for improvements to existing systems provide the ability for simultaneous multiplexed protein determinations in the same sample. These systems determine one protein at a time with a proportional increase in analysis time and sample volume, as well as changes in reagents, for additional analytes. There thus remains a need to make sensor arrays capable of measuring collections of proteins or bacteria, for example, simultaneously, without compromising analysis time or sample volume compared to that required for a single analyte.

The high electrical conductivity, excellent chemical stability, and unique structural robustness of carbon single wall nanotubes (SWNTs) have sparked considerable scientific and technological interest. The high electronic conductivity per unit mass suggests that carbon nanotubes (CNT) have the ability to facilitate direct electron-transfer with biomolecules, acting as molecular-scale electrical conduits, and providing opportunities for designing nano-scale immunosensors. Similarities between the size scales of enzymes and chemically shortened SWNTs may promote the likelihood of SWNTs to come within electron tunneling distance of enzyme redox sites, improving sensitivity for enzyme labels that generate signals by direct electron exchange with nanotubes. A number of immunosensor applications have been evaluated by utilizing electrochemistry of proteins, redox cofactors or DNA on flat mat-like layers of single or multi-walled carbon nanotubes. There remains a need for improvement in immunosensor applications of carbon nanotubes.

### SUMMARY

An immunoassay device comprises a plurality of carbon nanotubes having a first end and a second end, wherein the nanotubes are aligned substantially parallel relative to one another; a substrate responsive to an electrochemical signal, the substrate being attached to the first end of at least a portion of the plurality of nanotubes; and a capture antibody attached to at least a portion of the nanotubes not at the first end.

An array comprises one or more immunoassay devices disposed on a support.

An immunoassay method comprises providing the disclosed immunoassay device, contacting the immunoassay device with a test sample under conditions suitable for binding of an analyte to the capture antibody, wherein binding of the antigen generates, directly or indirectly, an electrochemical signal and detecting the signal.

A method of making an immunosensor, comprises disposing a first end of a plurality of carbon nanotubes onto a substrate responsive to an electrochemical signal, wherein the nanotubes are aligned substantially parallel relative to one another; and attaching a capture antibody to at least a portion of the nanotubes.

### BRIEF DESCRIPTION OF THE DRAWINGS

Referring now to the Figures, which are exemplary embodiments, and wherein the like elements are numbered alike:

FIG. 1 illustrates an embodiment of the assembly of SWNTs on a substrate.

FIG. 2 is an AFM image of a finished SWNT forest.

FIG. 3 is a conceptual depiction of horseradish peroxidase (HRP)-linked sandwich assay of biomarker protein PSA using a SWNT amperometric immunosensor.

FIG. 4 is a schematic of a procedure for preparing multiple enzyme labeled CNTs with high HRP/Ab₂ ratios: (A) shortening and carboxyl-functionalization of multiwalled CNTs, and (B) simultaneous bioconjugation with multiple HRP molecules and anti-PSA secondary antibody (Ab₂).

FIGS. 5 and 6 shows the results for a mediated amperometric sandwich assay at −0.2 V and 2000 rpm for HSA as an analyte in which SWNT/anti-HSA immunosensors were incubated with 10 μL HSA solution. FIG. 6 shows the currents after placing electrodes in buffer containing 0.4 mM hydroquinone mediator, then injecting H₂O₂ to 0.4 mM. FIG. 6 shows the influence of HSA concentration on steady state current for a SWNT/anti-HSA immunosensor (n=4).

FIGS. 7 and 8 show the results of mediated amperometric sandwich assays at −0.2 V and 2000 rpm for PSA in which SWNT/anti-PSA immunosensors were incubated with 10 μL serum containing PSA. Current was developed by placing sensors in buffer containing 0.4 mM hydroquinone mediator, then injecting H₂O₂ to 0.4 mM. FIG. 7 shows the results after using 10 μL 0.6 nmol mL⁻¹ anti-HSA-HRP for 1 hr (measured DL 10 Fmol mL⁻¹, 0.4 ng mL⁻¹). FIG. 8 shows the results after using CNT-HRP-Ab₂ with HRP:Ab₂ about 300 (measured DL 0.25 Fmol mL⁻¹, 0.01 ng mL⁻¹). Controls are shown on right in each graph: (a) SWNT-anti-PSA immunosensor with no PSA, (b) anti-PSA treated bare PG electrode and (c) anti-PSA treated bare PG electrode with iron oxide-Nafion coating.

FIG. 9 shows the influence of PSA concentration in 10 μL serum on steady state current for SWNT/anti-PSA immunosensors in assays using conventional HRP-Ab₂ (n=4).

FIG. 10 shows the influence of PSA concentration in 10 μL serum on steady state current for SWNT/anti-PSA immunosensors in assays amplified by using CNT-HRP-Ab₂ conjugates with HRP/Ab₂ about 300.

FIG. 11 shows a CNT forest disposed on a gold grid.

FIG. 12 shows an embodiment of an array of electrodes.

### DETAILED DESCRIPTION

Described herein are immunosensors comprising a plurality of CNTs disposed on a substrate. The immunosensors provide a generic platform wherein a wide range of electrochemical immunoassays can be integrated onto chip-based arrays. The immunosensors may be employed in a versatile, miniature array format for immunoassays capable of determining multiple analytes such as proteins or pathogenic bacteria in a single sample. In one embodiment, the immunosensors are suitable for use in a peroxidase-linked immunoassay.

Conductive, patternable, carbon nanotubes are suitable building blocks for amperometric micro- and nano-scale biosensor arrays. Carbon nanotube forests can be deposited or grown at specific locations in predetermined patterns. Another advantage of the carbon nanotube forests is that all the nanotubes point up toward the attached antibodies, increasing the probability of close contact between nanotubes and redox centers. The fact that carbon nanotube forests can directly exchange electrons with biomolecules such as enzymes, serving as molecular wires, simplifies sensor construction since electron-transfer mediating materials are minimized while achieving high sensitivity and low detection limits.

Based on these principles, the immunosensors described herein comprise a plurality of carbon nanotubes attached at a first end to a substrate responsive to an electrochemical signal, together with a capture antibody attached to at least a portion of the carbon nanotubes that is not attached to the substrate. Binding of the capture antibody to an antigen can be detected via an electrochemical signal that is transmitted to the substrate responsive to the signal. The electrochemical signal can be generated directly by the antigen-capture antibody interaction, or indirectly via the interaction of the antigen with an electrochemical detector such as a secondary antibody conjugated to a molecule capable of producing a signal that can be detected by an electrochemical method.

The disclosed biosensor comprises a substrate responsive to an electrochemical signal onto which a plurality of carbon nanotubes having a first end and a second end are assembled. The nanotubes are aligned substantially parallel relative to one another so that the substrate responsive to an electrochemical signal is attached to the first end of at least a portion of the plurality of nanotubes. The carbon nanotubes are substantially perpendicular to the substrate. Suitable substrates responsive to an electrochemical signal include electrodes. The term “electrode” refers to an electrical conductor that conducts a current in and out of an electrically conducting medium. The electrode may be present in the form of an array, consisting of a number of separately addressable electrodes. The electrode comprises an electrically conductive material. For example, gold, copper, carbon, tin, silver, platinum, palladium, indium tin oxide (ITO) or combinations comprising one or more of the foregoing materials may be employed. Among these materials, because of excellent electrical conductivity and chemical stability, gold electrodes, carbon electrodes, and tin oxide are preferable, and carbon electrodes are most preferable. In one embodiment, the electrode is in the form of a layer. It is to be understood that as used herein, a “layer” may have a variety of configurations, for example rectangular, circular, a line, an irregular dot, or other configuration. A suitable electrode is a pyrolytic graphite disk (PG) such as that available as PG from Advanced Ceramics.

Optionally, the substrate further comprises a conductive polyion to improve amperometric sensitivity of the sensor. One or more layers of conductive polyion can be disposed on the substrate. Nafion® or sulfonated polyaniline (SPAN), for example, can be employed to “wire” the proteins to conventional graphite electrodes. SPAN is self-doped and electroactive in the medium pH range where enzymes have maximum activity. The water solubility of SPAN makes it compatible with alternate layer-by-layer electrostatic self-assembly. Layers of SPAN (e.g., about 50% sulfonated on benzene rings) can be made on rough pyrolytic graphite (PG) electrodes. Then, stable electroactive films may be grown layer-by-layer on the underlayers of SPAN, featuring layers of antibody assembled with alternating layers of poly(styrene) sulfonate.

After deposition of the optional conductive polymer and prior to deposition of the carbon nanotubes, the substrate may optionally be treated to facilitate attachment of the carbon nanotubes. The surface may, for example, be treated with FeCl₃ to form Fe(OH)ₓ precipitates on the substrate surface. The layer of Fe(OH)ₓ may be formed by immersion of the electrode in an aqueous solution of FeCl₃. Other suitable substrate surface treatments include amine treatment and treatment with 1-(3-(dimethylamino)propyl)-3-ethylcarbodiimide hydrochloride (EDC), for example.

The carbon nanotubes, single walled or multi walled, may optionally be acid functionalized prior to deposition. Acid functionalization can be accompanied by oxidative shortening. Acid functionalization (i.e., carboxy functionalization) of carbon nanotubes can be accomplished by incubating the carbon nanotubes in acid for a time and at a temperature sufficient to produce the desired level of acid functionalization in the population of carbon nanotubes. Acid functionalization may optionally be accompanied by and/or followed by sonication. Suitable acids are mineral acids such as H₂SO₄, HNO₃, and combinations comprising one or more of the foregoing acids. A suitable acid functionalization protocol is treating SWNTs with a (7:3) mixture of HNO₃:H₂SO₄, for 6 hours at temperatures of about 40° C. to about 100° C., specifically about 40° C. to about 60° C. Alternative means of introducing carboxy functionalization include, for example, treatment with oxygen (at elevated temperatures, e.g., at about 400° C.), or treatment with hydrogen peroxide (e.g., at about 40° C. to about 100° C.).

Suitable suspending solvents for use with acid functionalized carbon nanotubes include polar solvents such as, for example, dimethylformamide (DMF), dimethylacetamide (DMAC), formamide, methyl formamide, hexamethylenephosphormamide, dimethylsulfoxide (DMSO), and combinations comprising one or more of the foregoing suspending solvents.

In one embodiment, the carbon nanotube forest assembly process involves the self-assembly of oxidatively shortened SWNTs onto the substrate responsive to a signal. Monolayers of vertically aligned, shortened SWNTs are assembled from DMF dispersions onto the substrate. In one embodiment, nanotubes are carboxyl-functionalized and shortened by sonication in 3:1 HNO₃/H₂SO₄ for 4 hours at 70° C. These SWNTS are filtered, washed with water and dried before dispersing in DMF.

The carbon nanotubes to be assembled onto the substrate may be of a suitable functional length, for example about 1 to about 100 nm long, specifically about 20 to about 30 nm long. SWNTs, for example, typically have individual diameters of about 1.4 nm. The “forests” comprise a plurality of nanotubes. Bundles having a suitable functional largest diameter may be used, for example bundles having a largest diameter of about 3 to about 1000 nm or more, or more specifically about 30 to about 200 nm may be used. The bundles may have a regular or irregular outline. In one embodiment, the nanotubes are deposited or self-assembled onto the substrate in a predetermined pattern. Other techniques, such as nanolithographic techniques, (e.g., electron beam lithography, together with appropriate masks), may be used to provide appropriate patterning, e.g., in 50 by 50 micrometer arrays.

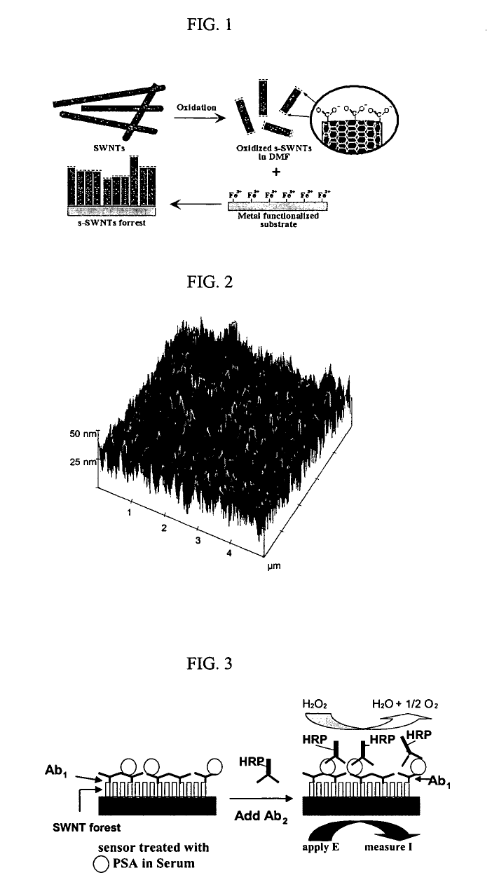

After immersion of the substrates into DMF dispersions of shortened carbon nanotubes, for example, vertically aligned assemblies of nanotubes are formed (e.g., SWNT forests), which may then be dried in vacuum. FIG. 1 illustrates an embodiment of the assembly of SWNTs on a substrate and FIG. 2 illustrates a finished SWNT forest.

In one embodiment, shortened carbon nanotubes (e.g., SWNTs) are aged in DMF dispersions prior to deposition on the substrate so that defects are largely removed making the sidewalls more hydrophobic and leading to formation of much more dense SWNT assemblies. These defects are believed to originate from counter ions balancing the positive charge of the oxidized (P-doping) SWNTS. The basicity of DMF dispersions controls the time necessary for D-doping. D-doping removes the counter ions from the nanotubes. Suitable aging times are for example, on day to six months. Nearly complete coverage of the underlying substrate with nanotubes of very high conductivity was achieved by aging the SWNT dispersions for 3 months prior to deposition.

Several factors may come into play to produce a successful assembly of carbon nanotubes on a substrate. The driving force for the assembly may be acid/base neutralization between iron hydroxides deposited on the substrate surface and the carboxylic acid groups of functionalized carbon nanotubes. Since carboxylic acids can be deprotonated by various metal oxides the carbon nanotube assembly process may also be promoted by Coulombic forces between the carboxylate anion headgroup and iron oxides coated on the substrate. These carbon nanotube forests possess significantly higher packing density and thus superior mechanical properties than vertical carbon nanotubes grown by chemical vapor deposition.

In one embodiment, the carbon nanotube forest assembly process involves the self-assembly of oxidatively shortened SWNT onto the substrate responsive to a signal, for example a layer of Fe³⁺-Nafion® or of SPAN on an electrode, or iron hydroxide nanoparticles on pyrolytic graphite (PG) electrodes.

Carbon nanotubes greatly increase the surface area of traditional 2-D electrodes while maintaining high conductivity and providing surface functional groups for bioconjugation with bioactive molecules such as enzymes and capture antibodies. In one embodiment, the bioactive molecule comprises a capture antibody represented herein as Ab₁. A variety of bioconjugation techniques may be employed, including adsorption and covalent bonding. For example, terminal carboxylate groups on carbon nanotube forests enable covalent binding of nanotubes with proteins through amide linkages, thus coupling sensing biomolecules to transducers. Water-soluble carbodiimides such as 1-(3-(dimethylamino) propyl)-3-ethylcarbodiimide hydrochloride (EDC) and N-hydroxysulfosuccinate (NHSS) can be used to facilitate binding of bioactive molecules to CNTs. Other, bioconjugation reactive groups can be employed, including, but not limited to amines, maleimide, thiols, NHS esters, and the derivatives of these reactive groups. N,N′-dicyclohexylcarbodiimide (DCC) may also be employed in bioconjugation strategies. The capture antibodies should provide maximum coverage on the SWNT forest as is consistent with high sensitivity of the sensors. In one embodiment, the capture antibody is disposed on the end of the carbon nanotubes.

In one embodiment, a suitable methodology for deposition of a bioactive molecule on a CNT forest involves placing μL-sized drops of 0.4 M EDC/0.1 M NHSS in water on SWNT forest surfaces for 10 minutes, washing with water, adding a drop of 0.5 mg/mL capture antibody in pH 7 buffer, and reacting for several hours before a final wash. Such procedures are readily automated using robotics.

For development and fine tuning of capture antibody (Ab₁) attachment to the SWNTs, relative efficiency may be assessed in three ways: (a) subsequent saturation binding of the antigen then HRP-Ab₂ (in sandwich assays), and an optical absorbance assay of the HRP activity on the surface for oxidation of o-phenylenediamine to 2,3-diaminophenazine; (b) rotating disk amperometric assay of the HRP activity for the saturated Ab1/antigen/HRP-Ab₂ surfaces, using optimal H₂O₂ concentration to achieve high sensitivity and low impact on film stability; and (c) weighing of each attached layer on SWNT forests built on quartz microbalance crystals (QCM).

Amperometry and QCM may also be employed to estimate binding constants to evaluate and choose antibodies for new analytes. Signal vs. concentration of antigen or HRP-Ab₂ is measured on sensors and QCM crystals from detection limit to saturation, and these data will be fit to the Langmuir adsorption isotherm appropriate for adsorption of molecules onto surfaces. This will provide effective binding constants (K_{B}) for antigen to Ab₁ by varying antigen concentration and for HRP-Ab₂ by saturating with antigen and varying HRP-Ab₂. The Langmuir isotherm will be used in the form
q=K_{B}C/(1+K_{B}C) (1)
where θ is fractional surface coverage of the binding molecule that is obtained from the signals less the NSB control, and C is the concentration of the binding substance in solution. In the case of amperometry, θ may be taken as the ratio of the blank-corrected steady state current at C to that at saturation values of C. Binding of test antigens follows the general shape of the Langmuir isotherm with the predicted linear regions at very low C. Measurements will reflect analytical protocols so as to estimate K_{B} as close as possible to sensor operating conditions. If choices of several antibodies are available for a given analyte, those with the largest K_{B}'s may be chosen.

A quartz crystal microbalance (QCM) may be used to measure the amounts of Ab₁ attached, as well as bound antigen, and bound HRP-Ab₂. For these studies, SWNT films may be built on gold-coated quartz QCM resonators coated with mercaptopropylalcohol/mercaptopropionic acid (7/3), which has been used previously to mimic carbon surfaces. QCM resonators (9 MHz, AT-cut, International Crystal Mfg. Co.) with gold electrodes (0.16 cm²) will be used for measurements with reproducibility±1 ng. The desired layer will be built on the resonator, the film dried in dry nitrogen, and the frequency change measured at each stage of fabrication. The Sauerbrey equation for dry films gives the relation between adsorbed mass and frequency shift ΔF (Hz) in the absence of viscoelasticity changes. For 9 MHz quartz resonators, film mass/unit area (M/A, g cm⁻²) is:
M/A=−ΔF(1.83×10⁸) (2)
for gold electrodes of A=0.16±0.01 cm² on one side. The nominal thickness (d) of dry films can be estimated from an expression validated by high resolution SEM cross-sectional images of protein films:
d(nm)≈−(0.016±0.002)ΔF(Hz) (3)

Atomic force microscopy (AFM) may be employed to image layer appearance at one or more steps of film assembly. AFM will also reveal surface density and size features of the metal nanoparticles in the underlayer used to “stand up” the carbon nanotube forests. Atomic Force Microscopy (AFM) images of SWNT forests with HRP and biotin antibody attached reveal smoother contours compared to the “spiky” appearance of SWNT forests. After proteins are coupled onto the SWNT forests, the globular appearance of the coating in the AFM images is reminiscent of protein-polyion aggregates on macroscopic surfaces. There is increased height of protrusion and a widened domain compared to the SWNT forests before protein attachment, consistent with a thin layer of protein attached on top of the nanotube forests.

Suitable bioactive molecules for use in the biosensors include enzymes that participate in electrochemical reduction pathways such as those involving peroxides. Nonlimiting examples of suitable enzymes include horseradish peroxidase and myoglobin.

Suitable capture antibodies are those that are useful for the immunological detection of an antigen of interest. By way of example, but not limitation, anti-biotin, anti-human serum albumin (anti-HSA) and anti-prostate specific antigen (anti-PSA) can be employed as capture antibodies on carbon nanotube forests. Mouse immunoglobulin (IgG) can be employed as a control surface to assess non-specific binding independently. Mouse IgG provided a related immunoglobulin composition on the surface as the antibodies except it has no binding sites specific for the antigens. Other antibodies, such as those for detecting cancer biomarkers (obtainable, for example, from the Cancer Genome Anatomy Project, NIH, Bethesda) and those suitable for ELISA assays may be employed.

Suitable analytes for detection by the disclosed biosensors include antigens detectable by an antibody which can be attached to a CNT. Suitable antigens for detection by the disclosed biosensors include, for example, cancer biomarkers such as prostate specific antigen (PSA), platelet factor 4 (PF4), matrix metalloproteinase-2 (MMP-2), prostate-specific membrane antigen (PSMA), and combinations comprising one or more of the foregoing cancer biomarkers. Cancer biomarkers are typically proteins that can be objectively measured and evaluated as an indicator of cancer. Families of biomarkers for prostate and breast cancer, for example, have been developed. Different types of cancers can have distinctly different sets of biomarkers. An advantage of the disclosed biosensors is that an array of biosensors can be employed to detect a plurality of biomarkers on one chip. Detection of cancer biomarkers can be used to screen for particular types of cancers and is useful as an early detection strategy. Rapid detection of cancer biomarkers is also useful during cancer surgery to detect the spread of cancer biomarkers into surgical borders. Detection of cancer biomarkers can also be used in pathology such as in the analysis of lymph node tissue.

Additional suitable antigens for detection include proteins and peptides such as, for example, human IgG, human IgM, human serum albumin (HSA) and hormones such as human chorionic gonadotropic hormone. Examples of infectious agents that could be detected with immunoarrays include Salmonella, E. coli, anthrax, botulism, herpes and influenza viruses, and HIV retrovirus.

Samples such as serum and tissue samples can be contacted with the disclosed biosensor. Other suitable samples include water, aerosols and food. If an antigen which binds the capture antibody attached to the biosensor is present, the antigen should bind to the attached capture antibody. Detection of the antibody-antigen complex can be done in a number of ways.

One concern in the development of the disclosed biosensors is the reduction of non-specific binding (NSB). One objective is to develop surfaces and conditions to keep NSB of all biomolecules in the samples at ultra-low levels to improve detection limits for antigens. NSB may be reduced by employing 0.1 to 0.01% Tween 20 with BSA or casein at 0.5 to 2% levels. For example, pre-adsorption of 2% BSA and 0.05% Tween 20 onto the SWNT/anti-biotin surfaces decreased NSB to <0.2%.

A method of detecting an analyte comprises providing the disclosed biosensor, contacting the biosensor with a test sample under conditions suitable for binding of the analyte to the capture antibody, wherein the contacting generates, directly or indirectly, a signal and detecting the signal. Detecting is preferably performed by electrochemical means. In one embodiment, the analyte comprises an antigen such as a cancer biomarker. Detecting comprises, for example contacting the biosensor with a detector.

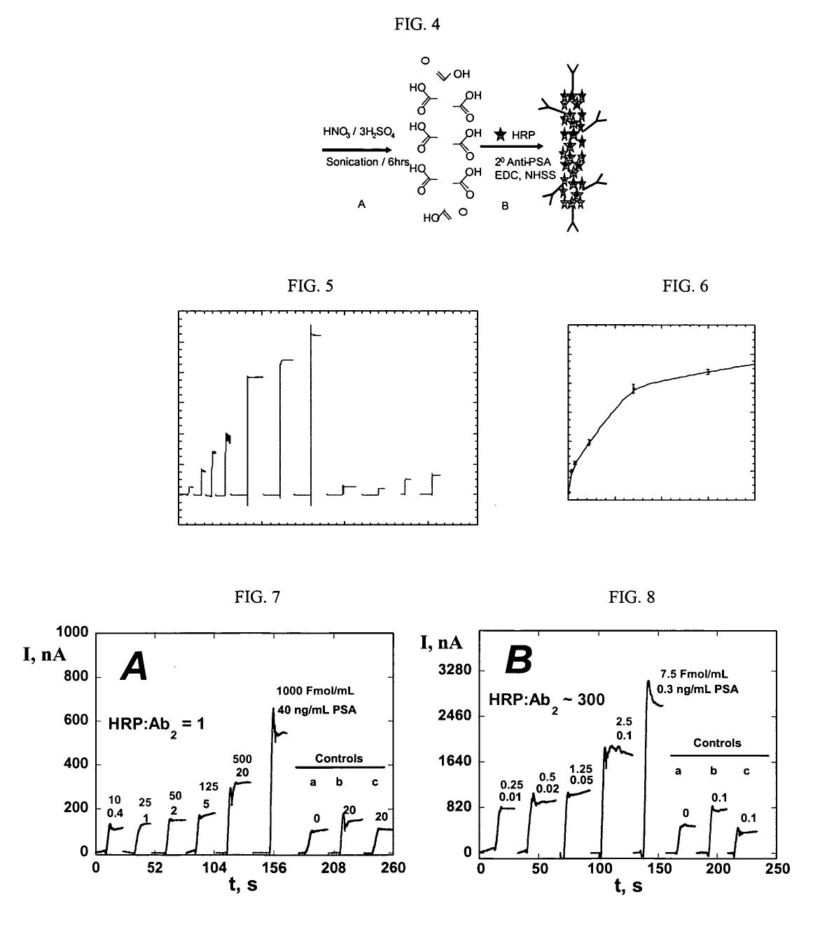

In one embodiment, a sandwich immunoassay format is used in which the detector molecule comprises an enzyme such as horseradish peroxidase (HRP) conjugated to the second any antibody used to form the sandwich. (See FIG. 3.) Suitable detectors include, for example, a secondary antibody conjugated to an enzyme such as HRP. In another embodiment, the detector comprises a nanostructure comprising a plurality of copies of both secondary antibody and horseradish peroxidase coupled thereto. Suitable nanostructures include single walled carbon nanotubes, multiwalled carbon nanotubes, conductive nanocrystals, carbon nanoropes, semiconducting nanowires, or a combination comprising one or more of the foregoing nanostructures. In one embodiment, the nanostructure is a multiwalled carbon nanotube. (See FIG. 4). The nanostructure detectors can be formed by oxidizing multiwalled carbon nanotubes with acid and ultrasound to make shortened carboxyl-derivatized CNTs. The protocol results in side walls of shortened multiwalled carbon nanotubes (e.g., 5-30 nm) with carboxylate groups. These carboxylate groups can be used to link multiple copies of HRP and antibodies to the nanotubes via amide linkages with the EDC/NHHS attachment protocol as described previously for attachment of the capture antibody to the nanotube forests. Attachment of the enzymes and antibodies make the nanotube conjugates water-soluble. An advantage of the carbon nanotube detectors is that a high ratio of HRP:secondary antibody can be employed. Suitable ratios of HRP:secondary antibody are 2000:1 to 100:1. The presence of multiple HRP molecules per secondary antibody greatly increases amperometric sensitivity. The detectors can be optimized by determining the optimum length by controlling the oxidation time and conditions, and assessing the optimum HRP/Ab₂ ratio by varying the protein concentrations in the conjugate reaction mixture.

Suitable electrochemical detection methods include, for example, capacitance and electrical impedance measurements. Detection methods include amperometry, voltammetry, surface plasmon resonance, and quartz crystal microbalance. The detector may use as signal generator an enzyme, which induces a change of ionic concentration, charge density, or electrochemical potential via enzymatic conversion of substrate, and produces an electrochemical change as signal

Suitable enzyme labels for detection include alkaline phosphatase (AP) and horseradish peroxidase (HRP). In general, a desirable enzyme should be able to efficiently catalyze an electron transfer reaction of a suitable mediator in the presence of a substrate for the enzyme.

Binding of an analyte specific to the capture antibody determines the quantity of detector molecule at the electrode surface (and hence the amount of current generated by the electrochemical reaction involved in the assay), thus permitting the quantitation of the analyte of interest. Alternatively, a competitive immunoassay format can be used in which the enzyme horseradish peroxidase (HRP) is conjugated to the analyte. In this case the analyte and the analyte HRP conjugate compete for a limited number of binding sites on an antibody immobilized electrode surface. Due to the competitive nature of the assay, the amount of surface bound analyte-enzyme conjugate (and hence the amount of current generated by the electrochemical reaction involved in the assay) is inversely proportional to the concentration of the analyte in the sample.

The surface bound HRP conjugate is detected by adding hydrogen peroxide and optionally a mediator. The mediator can facilitate electron transfer between the carbon nanotubes and the detector. The activity of the enzyme is determined electrochemically by the reduction of an electron transfer mediator. Examples of mediators that may be used include ferrocene and its derivatives, hydroquinone, benzoquinone, ascorbic acid or 3,3′,5,5′ tetramethylbenzidine (TMB).

An immunosensor or plurality of immunosensors may be provided in the form of an array. The array may be present, for example, on a solid support such as a chip. By “solid support” is meant a material that can be modified to contain discrete individual sites (including wells) appropriate to the formation or attachment of electrodes. The substrate may be a single material (e.g., for two dimensional arrays) or may be layers of materials (e.g., for three dimensional arrays). Suitable solid supports include metal surfaces such as glass and modified or functionalized glass, fiberglass, teflon, ceramics, mica, plastic (including acrylics, polystyrene and copolymers of styrene and other materials, polypropylene, polyethylene, polybutylene, polyimide, polycarbonate, polyurethanes, Teflon® and derivatives thereof, and the like), GETEK (a blend of polypropylene oxide and fiberglass), and the like, polysaccharides, nylon or nitrocellulose, resins, silica or silica-based materials including silicon and modified silicon, carbon, metals, inorganic glasses, a variety of other polymers, and combinations comprising one or more of the foregoing materials. An exemplary array is shown in FIG. 12.

Arrays will allow the determination of many analytes at once in replicate assays for a single sample. For example, arrays can be used to detect a selection of cancer biomarker proteins for diagnostics applications, or a selection of pathogenic bacteria in public health or biohazard applications.

Elements in the array may also feature different antibodies in replicate to increase analytical reliability. The protocols developed for constructing SWNT forests provide excellent versatility for array fabrication and miniaturization. All the steps are solution processable at room temperature, and should be amenable to automated fabrication. Deposition of the nanotubes in the forest arrangement onto thin conductive polymer-iron oxide layers provides conductive, patternable carboxylate functionality for antibody attachment. Certainly other methods of antibody attachment to arrays are possible, e.g., the use of functionalized alkylthiol layers on gold array elements. However, SWNT forests are stable over a wide range of applied potentials and provide a high surface area, carboxylated surface ready for high-coverage chemical linkage with antibodies.

All fabrication steps are compatible with, for example, the MicroSys 4000 spotter, which can dispense droplets of 20 nL to 4 μL rapidly in a computer-controlled predesigned pattern with a reproducibility of ±6% at the lower volume range. Precision of spot location is ±2 μm. These characteristics are suitable for antibody attachment on a 50 μm electrode arrays, for example. The spotting device may be equipped with the capability to wash the electrodes several times after every step of the element fabrication, e.g. by spotting the electrodes with water or another appropriate solvent, then removing the solvent with a mini-vacuum tube attached to the built-in vacuum system of the MicroSyn 4000.

A kit for screening or medical diagnostics, for example, includes one or more immunosensors as described herein. A plurality of immunosensors may be provided in the form of an array. The immunosensor or array of immunosensors may be provided on a solid support. The kit may include appropriate buffers, detection reagents and other solutions and standards for use in the methods described herein. In addition, the kits may include instructional materials containing directions (i.e., protocols) for the practice of the method(s). While the instructional materials typically comprise written or printed materials, they are not limited to such. A medium capable of storing such instructions and communicating them to an end user may be employed. Such media include, but are not limited to electronic storage media (e.g., magnetic discs, tapes, cartridges, chips), optical media (e.g., CD ROM), and the like. Such media may include addresses to internet sites that provide such instructional materials.

An instrument for performing toxicity screening is also included. The instrument can be designed for simple and rapid incorporation into an integrated assay device, e.g., a device comprising an electrochemical detector (e.g., voltammetry) circuitry, appropriate means for administration of a sample, and computer control system(s) for control of sample application, and analysis of signal output. The instrument is designed to employ an immunosensor as described herein. The instrument may be designed to employ a plurality of immunosensors in the form of, for example, an array. The immunosensor or array of immunosensors may be provided on a solid support. Automated or semi-automated methods in which the immunosensors are mounted in a flow cell for addition and removal of reagents, to minimize the volume of reagents needed, and to more carefully control reaction conditions, may be employed.

Flow-cell arrangements may also be employed and may be convenient for certain repetitive assays. A flow-injection system comprising a mini-pump with an injector and a Bioanalytical Systems thin-layer detector cell with the appropriate SWNT forest/antibody systems attached to the working electrode can be employed.

The invention is further illustrated by the following non-limiting examples.

In these examples, characterization of products was carried out using several techniques. A CHI 430 electrochemical workstation or a CHI 660 potentiostat was used for cyclic voltammetry and amperometry at ambient temperature (22±2° C.). A three electrode cell was used employing a saturated calomel reference electrode (SCE), a platinum wire as counter electrode and ordinary plane pyrolytic graphite as working electrode. (Advanced Ceramics, are of 0.2 cm²). The electrochemical buffer was pH 6.8 phosphate buffer, 0.1 M, 0.137 M NaCl and 2.7 mM KCI. The buffers were purged with purified nitrogen and a nitrogen environment was maintained in the cell during experiments. Amperometry was done at −0.2 V vs. SCE (Saturated Calomel Electrode) with the SWNT working electrode rotated at 2000 RPM, for optimum sensitivity unless otherwise stated. For atomic force microscopy (AFM), tapping mode measurements were performed on smooth Si(100) wafers with a Nanoscope IV scanning probe microscope. Resonance Raman spectra of SWNT forest assemblies on pyrolytic graphite electrodes were taken with a Renishaw Ramanscope 2000 using a 785 nm (1.58 eV) argon laser focused on a 1 μm spot by a 100× objective lens.

### EXAMPLE 1

### Assembly of SWNT Forests

SWNT Forests were assembled on Si wafers for AFM and Raman spectroscopy and on abraded basal plane pyrolytic graphite (PG) disk electrodes for sensing experiments. Nanotubes were carboxyl-functionalized and shortened by sonication in 3:1 HNO₃/H₂SO₄ for 4 hr at 70° C. These shortened nanotubes were filtered, washed with water, dried, and suspended in DMF. PG and Si surfaces were prepared for nanotube assembly by forming a bed of Nafion® on their surfaces onto which iron was adsorbed to later form a Fe(OH)ₓ surface precipitate. After immersion of these substrates into DMF dispersions of shortened SWNTs, vertically assemblies of nanotubes were formed (SWNT forests), which were then dried in vacuum for 18 hours.

Sensitivity is increased for H₂O₂, for example, by introducing prolonged aging time of SWNT dispersions in DMF prior to forest assembly. Resonance Raman spectra show clear differences between the assemblies made from SWNT dispersions aged for 1 hr and 3 months following the acid and sonication-assisted oxidation. The defect (D-band), typically observed between 1250 and 1450 cm⁻¹, which originates from the first-order scattering by in-plane hetero-atom substituents, grain boundaries, vacancies or the other defects and by finite size defects decreases when the SWNT/DMF dispersion are aged 3 months showed large decreases in D-band width compared to the SWNT/DMF dispersion aged 1 hr. Atomic force microscopy (AFM) images showed that SWNT forests made from the dispersions aged for 3 months achieved nearly full coverage of the underlying surface

### EXAMPLE 2

### Prototype Biotin Sensor Using SWNT Forests

The anti-biotin/biotin pair was chosen for initial evaluation of the feasibility of designing immunosensor assays on SWNT forests. First, the anti-biotin antibody was attached to SWNT forests on 0.16 cm² area PG disks. Use of N-hydroxysulfosuccinate (NHSS) along with EDC in a coupling cocktail followed by antibody addition gave 3-fold higher yields of covalently bound anti-biotin on the SWNT forests than just EDC alone. AFM images of the anti-biotin layer were similar to other protein layers on SWNT forests.

SWNT forests with bound anti-biotin were analyzed by rotating disk amperometry. Treatment of the SWNT/Ab₁ electrode with 2% BSA and 0.05% Tween 20 before the binding and measurement steps provided low non-specific binding of biotin-HRP. By including soluble hydroquinone as a mediator to shuttle electrons between HRP labels and the SWNT forests, the detection limit for biotin-HRP was about 2 picomol ml⁻¹ (0.1 ng/ml), corresponding to the detection limit of traditional ELISA. Non-specific binding in the mediated assay was estimated at about 0.1%, and the linear range was 2-75 pmol ml⁻¹.

SWNT/anti-biotin sensors were also evaluated in a competitive assay for unlabeled biotin using a hydroquinone mediator. The detection limit in this inherently less sensitive assay was 10 nmol ml⁻¹. Greatly improved detection limits using soluble redox mediators indicate that not all the HRP in the bound Ab/biotin-HRP is in direct electrical communication with the measuring circuit. These findings suggested that molecular wiring using redox polymers or conductive polymers are viable approachs to link all of the enzyme to the measuring circuit, and thereby to greatly improve sensitivity and detection limits.

### EXAMPLE 3

### Use of Conductive Polymers to Improve Sensitivity of Immunoassay Biosensors

It was suspected that the underlying bed of Nafion®-iron oxide forms a tiny resistive junction where the nanotubes contact the underlying pyrolytic graphite, and that this resistive junction may degrade sensor performance. By using SPAN instead of Nafion as the polymer glue to hold iron oxide nanoparticles onto the PG surface in the underlying bed, it was believed that the conductance of the micro-junctions between the nanotubes and the electrical contact graphite might be significantly increased. SWNT forests were thus constructed on such a SPAN-iron oxide bed, and tested in an amperometric sandwich assay for human serum albumin (HSA). Anti-HSA antibody was chemically attached to the SWNT forest by the EDC/NHSS protocol as described above, then the sensors were incubated with single drops of various concentrations of HSA, followed by washing, and incubation with a drop of HRP-labeled HSA antibody. The protocol of 2% BSA+0.05% Tween-20 was used to inhibit NSB. Amperometric currents were developed by injection of dilute H₂O₂. Steady state currents were readily measurable down to 15 pmol mL⁻¹ and below on these sensors. A control experiment consisting of all the steps above but omitting the HSA incubation gave average steady state current of 1 nA, which appears to result from residual non-specific binding. Measurement of the HSA detection limit taking into account this control gave 10 pmol mL⁻¹, or a mass detection limit of 0.1 picomol of HSA in the 10 μL droplet used. Calibration was linear from about 3000 to 10 μpmol mL⁻¹. Similar HSA sensors constructed with SWNT forests on a layer of the insulating polyion Nafion® and iron oxide instead of SPAN-iron oxide had detection limits of about 500 pmol mL⁻¹, demonstrating a 50-fold improvement by using SPAN for molecular wiring in these devices.

### EXAMPLE 4

### HSA Immunosensor Using a Soluble Electron Transfer Mediator

In order to improve electron transfer efficiency, electron transfer mediation by hydroquinone was explored. Voltammetry and amperometry showed that hydroquinone efficiently mediated the reduction of peroxide-activated HRP in the HSA sandwich assay at an optimum concentration of 0.4 mM. Immunosensors were treated with casein and detergent to minimize NSB. The rotating disk amperometry detection included both H₂O₂ and hydroquinone. The mediated steady state current increased (FIG. 5) with the increase in the amount of HSA in the concentration range from 1 to hundreds of pmol mL⁻¹ (nM). The calibration curve in this case was linear at concentrations of HSA less that about 20 pmol mL⁻¹ (FIG. 6), but the signal continued to increase up to several hundred pmol mL⁻¹. Compared to the unmediated case, sensitivity improved 10,000-fold to 46 nA/nM compared to the unmediated case.

Control experiments for the mediated detection of HSA (FIG. 6) demonstrate the gain in sensitivity afforded by SWNT forests. In control (a) a PG electrode coated with Nafion-iron oxide was treated with anti-HSA and exposed to full sandwich assay procedure using 140 pmol mL⁻¹ HSA. The response was 16-fold smaller that that of the SWNT immunosensor for 140 pmol mL⁻¹, and only a little larger that of control (b), a SWNT immunosensor taken through the full procedure without HSA. The latter control response reflects the residual NSB. The detection limit (DL) for HSA estimated as 3× the noise level above this control was 1 pmol mL⁻¹ (1 nM). Controls (c) and (d) were bare PG electrodes without SWNTs taken through the anti-HSA attachment and mediated immunoassay procedures and exposed to 2 different HSA levels. Signals of these controls were about 8-fold smaller than for the full immunosensor at the equivalent HSA concentrations.

### EXAMPLE 5

### PSA Immunosensors and CNT-HRP-Ab₂ Amplification

The prostate cancer biomarker PSA has been detected with very high sensitivity. A key to this achievement was the preparation of nanotubes conjugated with HRP and Ab₂ (CNT-HRP-Ab₂) with high HRP:Ab₂ ratios e.g., 300:1. Briefly, commercial multiwalled carbon nanotubes (CNT) were oxidized with acid and ultrasound to make shortened carboxyl-derivatized CNTs. Ab₂ and HRP were then attaching using a standard EDC/NHSS protocol. CNT-HRP-Ab₂ conjugates were centrifuged, washed and used in sandwich immunoassays. Using this approach, PSA detection limit (DL) was measured at 0.25 Fmol mL⁻¹, 0.01 ng mL⁻¹.

The SWNT sensors employed anti-human PSA monoclonal antibody. FIGS. 7 and 8, for example, compare sensor response using conventional HRP conjugated anti-human PSA monoclonal antibodies (HRP:Ab₂=1; FIG. 7) with the CNT-HRP-Ab₂ conjugates (HRP:Ab₂=300; FIG. 8). Mediated amperometric sandwich assays at −0.2 V and 2000 rpm for PSA in which SWNT/anti-PSA immunosensors (base PG disk A=0.16 cm²) were incubated with 10 μL serum containing PSA (concentrations in Fmol mL⁻¹ and ng/mL labeled on curves) for 1 hr, then washed with 2% BSA+0.05% Tween-20 in PBS. Current was developed by placing sensors in buffer containing 0.4 mM hydroquinone mediator, then injecting H₂O₂ to 0.4 mM for (FIG. 7) after using 10 μL 0.6 nmol mL⁻¹ anti-PSA-HRP for 1 hr (measured DL 10 Fmol mL⁻¹, 0.4 ng mL⁻¹); (FIG. 8) after using CNT-HRP-Ab₂ with HRP/Ab₂ about 300 (measured DL 0.25 Fmol mL⁻¹, 0.01 ng mL⁻¹). Controls are shown on right in each graph, given with PSA concentrations: (a) SWNT-anti-HSA immunosensor with no PSA, (b) anti-PSA treated bare PG electrode and (c) anti-PSA treated bare PG electrode with iron oxide-Nafion coating.

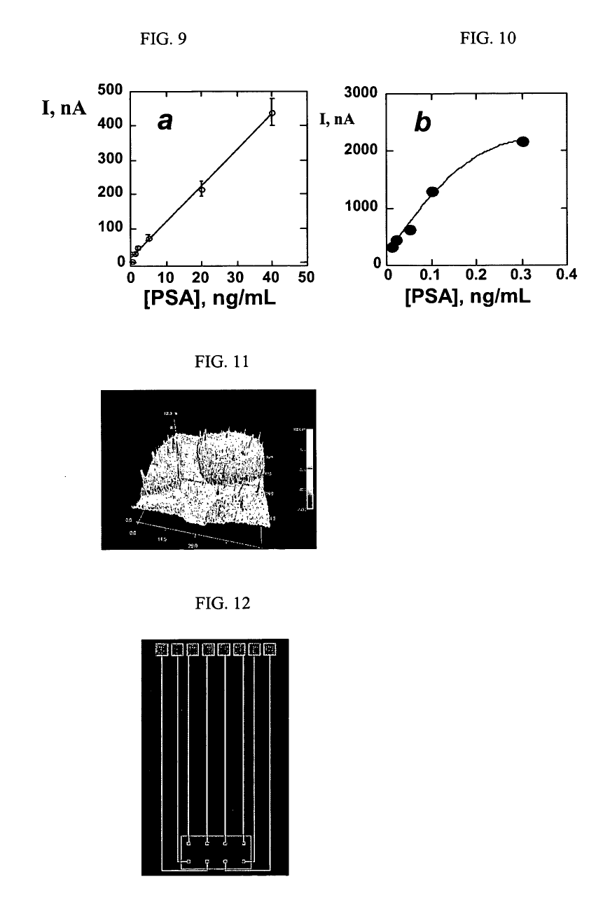

FIGS. 9 and 10 show the influence of PSA concentration in 10 μL serum on steady state current for SWNT/anti-PSA immunosensors: (FIG. 9) assays using conventional HRP-Ab₂ (n=4); (FIG. 10) assays amplified by using CNT-HRP-Ab₂ conjugates with HRP/Ab₂ about 300. Using hydroquinone as mediator provided DL about 25 Fmol mL⁻¹ (1 mg/mL) and sensitivity of about 440 nA/nM in the linear region for the CNT-HRP-Ab₂ (FIG. 10). The 30,000-fold better detection limit compared to the HSA immunoassay discussed above was achieved by using monoclonal antibodies and more dilute HRP conjugated secondary antibody to further decrease residual NSB. Again, the SWNT forests provided a significant gain in sensitivity over control immunosensors without nanotubes.

Replacing the usual secondary antibody enzyme conjugates with CNT-HRP-Ab₂ conjugates provided another 100-fold improvement in detection limit for PSA at 0.25 Fmol mL⁻¹, 0.01 ng mL⁻¹ (FIGS. 8 and 10). Further, comparison of the controls without nanotube to the full sensor configuration shows that the advantage of the SWNT forests was maintained. The zero PSA controls (labeled a in FIG. 7) suggest that nonspecific binding of the CNT-HRP-Ab₂ and the HRP-Ab₂ conjugates still control the DL, suggesting that further optimization is possible.

Sensitivities and DLs for PSA in buffer and serum were comparable, showing that the method is already amenable to sensitive detection in real samples. The CNT-HRP-Ab₂ as secondary antibody in the sandwich assay provides an exquisitely low DL. However, for PSA, the conventional HRP-Ab₂ provides an adequate detection limit and good linearity and reproducibility in the critical 4-10 ng/mL serum PSA range used for cancer diagnostics. Both methods should provide excellent utility.

### EXAMPLE 6

### Patterning of SWNT Forests

SWNT forests have been patterned on the micrometer size scale. Initial demonstrations employed Nafion coated on a Si wafer. A TEM grid was placed over this wafer and it was irradiated with an electron beam. This left a cross pattern of Nafion®. The usual iron oxide nanoparticle layer was then formed on the Nafion® pattern. Finally, SWNTs in DMF were deposited onto the patterned iron oxide. AFM images clearly showed the resulting SWNT forest pattern. This experiment demonstrates that the iron oxide precursor layer required underneath the SWNT forests can be deposited selectively on patterns of anionic polymer.

In studies directly relevant to array development, SWNT forest were patterned on gold array grids with spot diameters of about 30 μm (FIG. 11). With Au, deposition of Nafion® may not be not necessary to make the nanotube forests. The Au arrays were simply treated with aqueous FeCl₃, washed with HCl and DMF, and FeO(OH)/FeOCl nanoparticles formed on the surface. AFM showed that these nanoparticles formed selectively on the Au, suggesting an important role for the gold surface. The FeO(OH)/FeOCl nanoparticles formed the template pattern for deposition of SWNT forests from aged nanotubes dispersion in DMF. Most of the nanoparticles formed on the Au array elements, and very few on the Si underlayer (blue). Resonance Raman spectra (514 nm laser) measured at Au and Si regions respectively showed the G band (1592 cm⁻¹) characteristic of the carbon SWNTs was observed at Au regions but not at Si regions. This fabrication method could provide a simple basis for patterning SWNT forests on gold arrays for immunosensor development.

Carbon nanotubes forests perpendicularly aligned on pyrolytic graphite surfaces for amperometric peroxidase-linked immunoassays are disclosed. Immunosensors are made by attaching antibodies to the carboxylated ends of the nanotube forests. Utilizing direct electrochemistry of labels and additives to minimize non-specific binding, amperometric immunosensors achieved sub-nanomolar detection limits. Such ultramicroelectrodes may be used in the manufacture of multielement nanoimmunosensors and nanosensor arrays. These immunosensors may be used in applications such as proteomics and pathogen detection, as well as medical diagnostics.

Disclosed herein is a rapid, versatile, miniature array format for immunoassays capable of determining multiple analytes such as proteins or pathogenic bacteria in a single sample. As shown herein, conductive, patternable, SWNT are suitable building blocks for amperometric micro- and nano-scale biosensor arrays. Major practical advantages include high sensitivity and ultra-low detection limits for multiple analytes in minimal sample volume.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An immunoassay device comprising:
a plurality of carbon nanotubes having a first end and a second end, wherein the nanotubes are aligned substantially parallel relative to one another;
a substrate responsive to an electrochemical signal the substrate being attached to the first end of at least a portion of the plurality of nanotubes; and
a capture antibody attached to at least a portion of the nanotubes not at the first end.

2. The device of claim 1, wherein the nanotubes comprise single wall carbon nanotubes.

3. The device of claim 2, wherein the single wall carbon nanotubes are oxidatively shortened single wall nanotubes having a length of about 1 nm to about 100 nm.

4. The device of claim 1, wherein the substrate comprises an electrode.

5. The device of claim 4, wherein the substrate comprises a conductive polyion.

6. The device of claim 1, wherein the capture antibody is suitable to detect a cancer biomarker.

7. An array comprising one or more devices of claim 1 disposed on a support.

8. The array of claim 7 comprising at least two devices, each device having a different type of capture antibody attached thereto.

9. An immunoassay method, comprising
providing the immunoassay device of claim 1,
contacting the immunoassay device with a test sample under conditions suitable for binding of an analyte to the capture antibody, wherein binding of the analyte generates, directly or indirectly, an electrochemical signal and
detecting the signal.

10. The immunoassay method of claim 9, wherein detecting comprises contacting the device with a detector.

11. The immunoassay method of claim 10, wherein the detector comprises a secondary antibody conjugated to horseradish peroxidase.

12. The immunoassay method of claim 10, wherein the detector comprises a nanostructure comprising a plurality of copies of both secondary antibody and horseradish peroxidase coupled thereto.

13. The immunoassay method of claim 12, wherein the nanostructure comprises a single walled carbon nanotube, a multiwalled carbon nanotube, a conductive nanocrystal, a carbon nanorope, a semiconducting nanowire, or a combination comprising one or more of the foregoing nanostructures.

14. The immunoassay method of claim 12, wherein the ratio of horseradish peroxidase to secondary antibody is 2000:1 to 100:1

15. The immunoassay method of claim 10, wherein the capture antibody is suitable to detect a cancer biomarker.

16. The immunoassay method of claim 10, wherein detecting comprises adding hydrogen peroxide and an electron transfer mediator.

17. The method of claim 16, wherein the mediator comprises hydroquinone.

18. A method of making an immunosensor, comprising
disposing a first end of a plurality of carbon nanotubes onto a substrate responsive to an electrochemical signal, wherein the nanotubes are aligned substantially parallel relative to one another; and
attaching a capture antibody to at least a portion of the nanotubes.

19. The method of claim 18, further comprising, prior to disposing the first end of the plurality of carbon nanotubes, disposing one or more layers of conductive polyion on the substrate.

20. The method of claim 19, further comprising disposing FeCl₃ on the layer of conductive polyion.

21. The method of claim 19, wherein the substrate comprises an electrode.
